(19) United States
(12) Patent Application Publication
(10) Pub. No.: US 2018/0325615 A1
(43) Pub. Date: Nov. 15, 2018

(54) Remote Center of Motion Robotic Arm

(71) Applicant: Hiwin Technologies Corp., Taichung City (TW)

(72) Inventor: Hsuan-Tsung Chen, Taichung City (TW)

(21) Appl. No.: 15/594,735

(22) Filed: May 15, 2017

(51) Int. Cl. A61B 34/00; A61B 34/37; A61B 90/00

(52) U.S. Cl. CPC A61B 34/74; A61B 34/37; A61B 90/361; A61B 34/30; A61B 34/70; A61B 2034/743; A61B 90/37

(57) ABSTRACT

A remote center of motion robotic arm includes an arm unit, a drive unit and a control unit. The arm unit includes a base seat, a first link rotatable relative to the base seat, a mount seat, and a second link pivoted to the first link and rotatable relative to the mount seat. The drive unit includes first and second drive motors for rotating the first and second links. The control unit controls the drive unit to move the first and second links from an initial position to an objective position, such that the relative position between the base seat and the mount seat is maintained after the movement of the first and second links.

### FIELD

The disclosure relates to a robotic arm, and more particularly to a remote center of motion robotic arm.

### BACKGROUND

U.S. Pat. No. 8,167,872 discloses a conventional robotic arm for supporting an endoscope during an endoscopy procedure. However, the conventional robotic arm may easily obstruct an operating space for other medical instruments.

### SUMMARY

Therefore, an object of the disclosure is to provide a remote center of motion robotic arm that can alleviate the drawback of the prior art.

According to the disclosure, the remote center of motion robotic arm includes an arm unit, a drive unit and a control unit. The arm unit includes a base seat that has a first axial line, a first link that is mounted to the base seat and that is rotatable relative to the base seat about the first axial line, a mount seat that has a second axial line, and a second link that is pivoted to the first link, that is mounted to the mount seat, and that is rotatable relative to the mount seat about the second axial line. The drive unit includes a first drive motor that is connected to the first link and that is operable to drive rotation of the first link relative to the base seat about the first axial line, and a second drive motor that is connected to the second link and that is operable to drive rotation of the second link relative to the mount seat about the second axial line. The control unit is electrically coupled to the drive unit, and receives a control signal to control the first drive motor and the second drive motor to move the first and second links from an initial position to an objective position, such that the relative position between the base seat and the mount seat at the time that the first and second links are at the initial position and the relative position between the base seat and the mount seat at the time that the first and second links are at the objective position are substantially identical to each other.

### BRIEF DESCRIPTION OF THE DRAWINGS

Other features and advantages of the disclosure will become apparent in the following detailed description of the embodiment with reference to the accompanying drawings, of which:

FIG. 1 is a perspective view illustrating an embodiment of the remote center of motion robotic arm according to the disclosure;

FIG. 2 is a block diagram illustrating the embodiment;

FIG. 3 is a schematic perspective view illustrating a first link and a second link of the embodiment at an initial position;

FIG. 4 is a schematic view illustrating the first link and the second link at the initial position; and

FIG. 5 is another schematic view illustrating the first link and the second link at an objective position.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

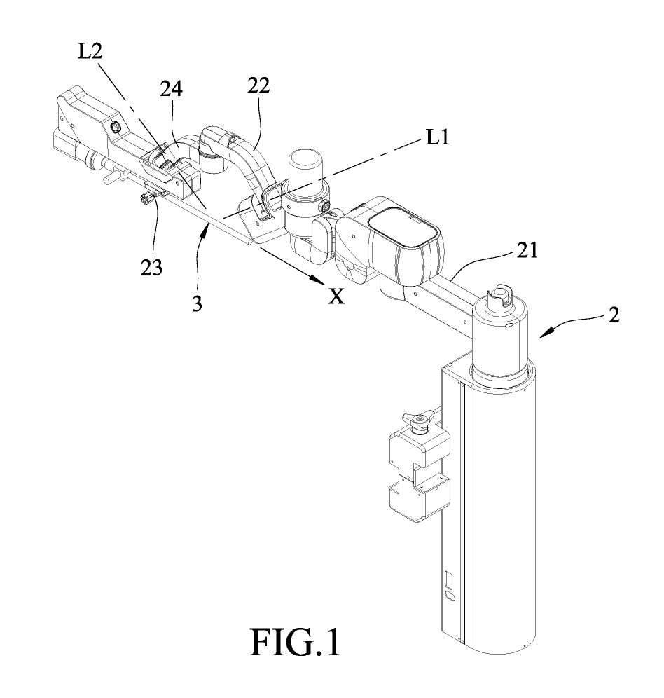

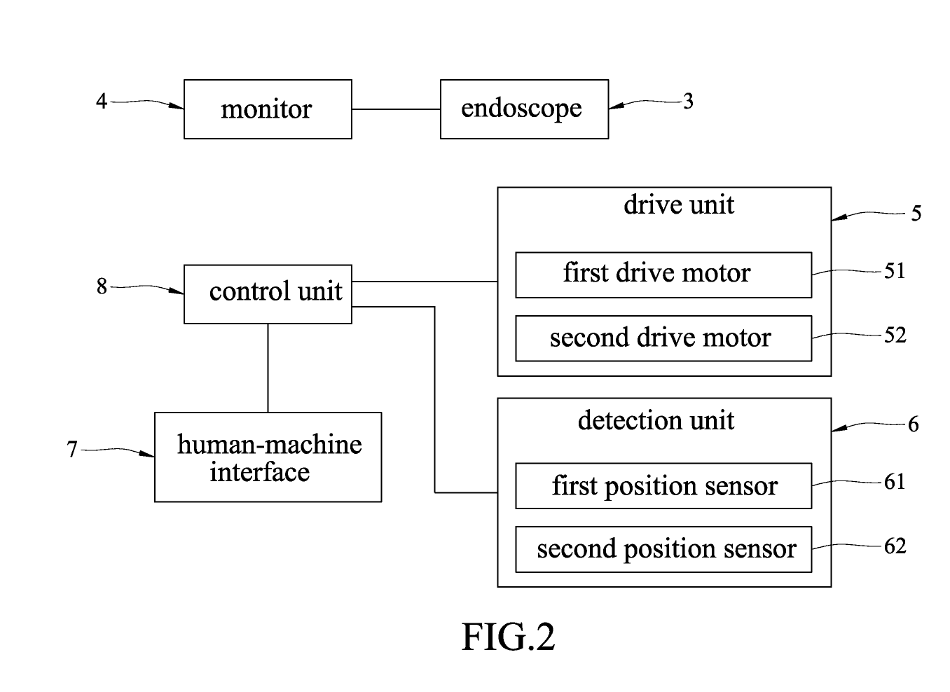

Referring to FIGS. 1 to 3, the embodiment of the remote center of motion robotic arm according to the disclosure includes an arm unit 2, an endoscope 3, a monitor 4, a drive unit 5, a detection unit 6, a human-machine interface 7 and a control unit 8.

The arm unit 2 includes a base seat 21 that has a first axial line (L1), a first link 22 that is mounted to the base seat 21 and that is rotatable relative to the base seat 21 about the first axial line (L1), a mount seat 23 that has a second axial line (L2), and a second link 24 that is pivoted to the first link 22, that is mounted to the mount seat 23, and that is rotatable relative to the mount seat 23 about the second axial line (L2).

The endoscope 3 is mounted to the mount seat 23, and is oriented in an objective direction (X) for taking images.

The monitor 4 is electrically coupled to the endoscope 3 for displaying the images taken from the endoscope 3.

The drive unit 5 includes a first drive motor 51 that is connected to the first link 22 and that is operable to drive rotation of the first link 22 relative to the base seat 21 about the first axial line (L1), and a second drive motor 52 that is connected to the second link 24 and that is operable to drive rotation of the second link 24 relative to the mount seat 23 about the second axial line (L2).

The detection unit 6 includes a first position sensor 61 that is connected to the first drive motor 51 for detecting a rotational position of the first drive motor 51, and a second position sensor 62 that is connected to the second drive motor 52 for detecting a rotational position of the second drive motor 52.

The human-machine interface 7 is for emitting a control signal. In one embodiment, the human-machine interface 7 is configured as a keyboard.

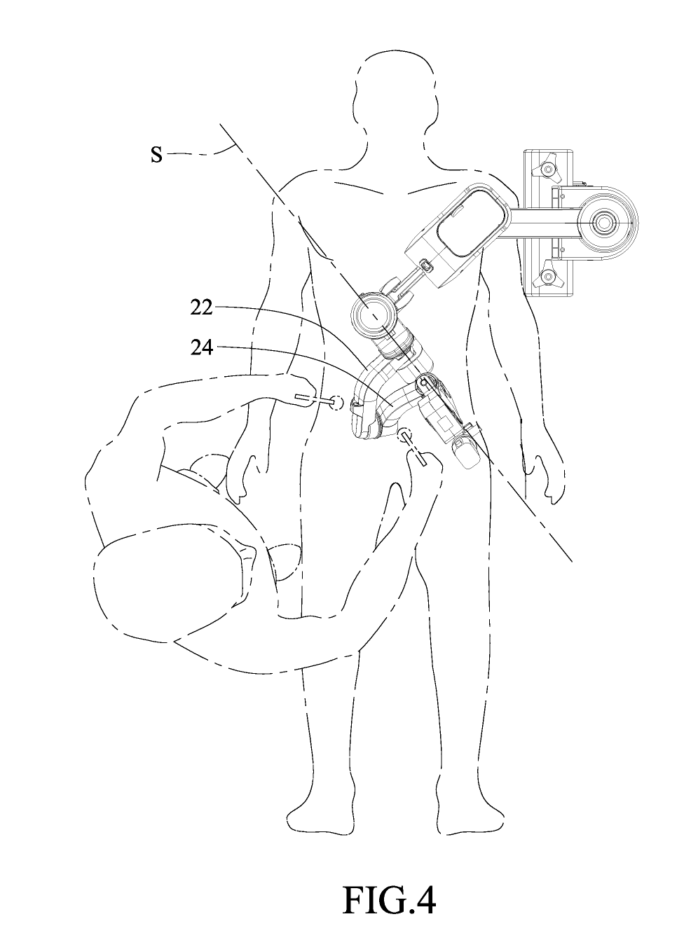

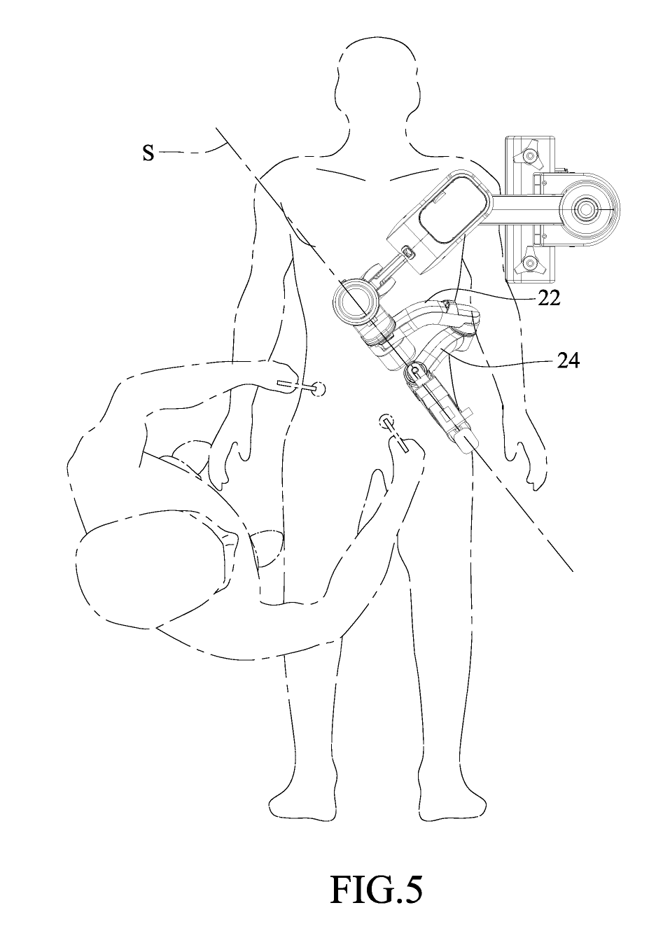

The control unit 8 is electrically coupled to the drive unit 5, the detection unit 6 and the human-machine interface 7, and receives the control signal emitted from the human-machine interface 7 to control the first drive motor 51 and the second drive motor 52 for moving the first and second links 22, 24 from an initial position (see FIG. 4) to an objective position (see FIG. 5). Note that, due to the characteristic of the remote center of motion robotic arm, the relative position between the mount seat 23 and the base seat 21 at the time that the first and second links 22, 24 are at the initial position and the relative position between the mount seat 23 and the base seat 21 at the time that the first and second links 22, 24 are at the objective position are substantially identical to each other.

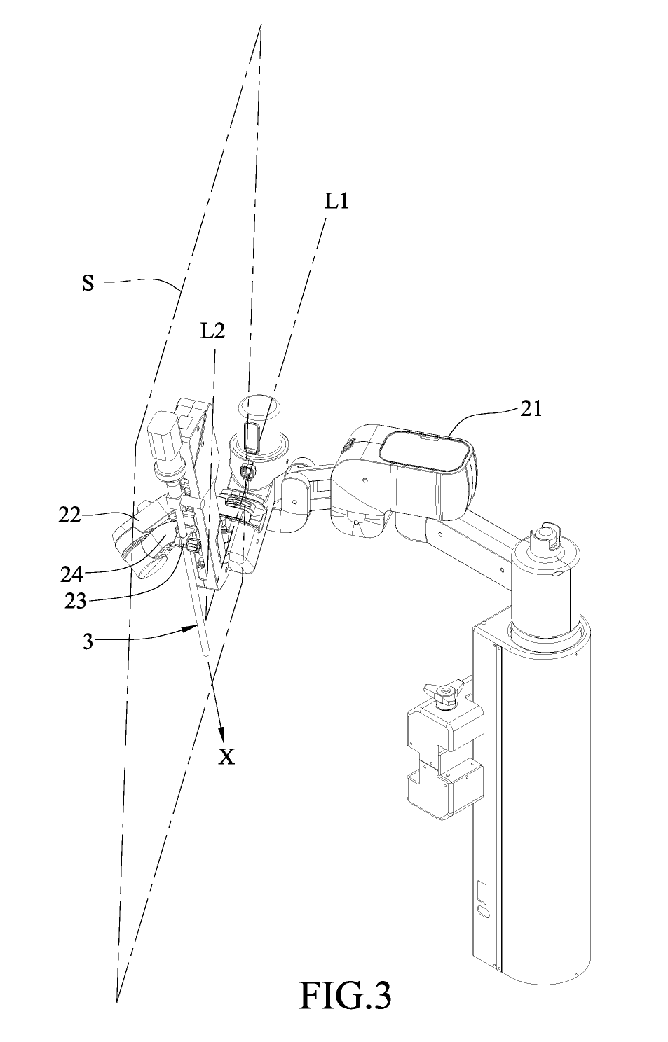

In this embodiment, both of the first axial line (L1) and the second axial line (L2) belong to an imaginary plane (S) (see FIG. 3). A joint between the first and second links 22, 24 (i.e., the pivoted point between the first and second links 22, 24) moves between two opposite sides of the imaginary plane (S) during the movement of the first and second links 22, 24 from the initial position to the objective position (see FIGS. 4 and 5). More specifically, the initial position of the first and second links 22, 24 and the objective position of the first and second links 22, 24 are symmetric to each other with respect to the imaginary plane (S) (i.e., the imaginary plane (S) serves as a plane of symmetry).

After the control unit 8 receives the control signal for moving the first and second links 22, 24 from the initial position to the objective position, the control unit 8 first obtains the rotational positions of the first and second drive motors 51, 52 that are respectively detected by the first and second position sensors 61, 62, and then calculates an initial relative position among the base seat 21, the first link 22, the second link 24 and the mount seat 23 accordingly, so as to determine the initial position of the first and second links 22, 24, and to determine the orientation (i.e., the objective direction (X)) of the endoscope 3.

Then, the control unit 8 calculates an objective relative position among the base seat 21, the first link 22, the second link 24 and the mount seat 23 where the position of the first and second links 22, 24 is symmetric to the position of the first and second links 22, 24 at the time that the first and second links 22, 24 are at the initial position with respect to the imaginary plane (S) and where the relative position between the base seat 21 and the mount seat 23 is maintained so as to determine the objective position of the first and second links 22, 24, and calculates necessary rotational movements of the first and second drive motors 51, 52 for moving the first and second links 22, 24 to the objective position with the relative position between the base seat 21 and the mount seat 23 being maintained.

Finally, the control unit 8 controls the first and second drive motors 51, 52 to drive movement of the first and second links 22, 24 from the initial position to the objective position.

In practical use, when the first and second links 22, 24 obstruct an operating space for other medical instruments so that work is interrupted, the control unit 8 is operable to control the first and second drive motors 51, 52 upon reception of the control signal emitted from the human-machine interface 7, so as to move the first and second links 22, 24 from the initial position to the objective position. As such, the first and second links 22, 24 are removed from the operating space for operation of the medical instruments, and the relative position between the base seat 21 and the mount seat 23 is maintained for the work to continue without interruption.

It should be noted that, in a variation, the mount seat 23 may be mounted with at least one of instruments different from the endoscope 3 according to actual demands.

In summary, since the first and second links 22, 24 are movable between the initial position and the objective position that are symmetric to each other with respect to the imaginary plane (S) and since the relative position between the base seat 21 and the mount seat 23 is maintained after the movement of the first and second links 22, 24 from the initial position to the objective position, the first and second links 22, 24 can be moved from the initial position to the objective position without eventually changing the relative position between the base seat 21 and the mount seat 23 and so as to not obstruct an operating space. Moreover, by virtue of the control unit 8, the drive unit 5 and the detection unit 6, the first and second links 22, 24 can be rapidly moved from the initial position to the objective position.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A remote center of motion robotic arm comprising:
an arm unit including a base seat that has a first axial line (L1), a first link that is mounted to said base seat and that is rotatable relative to said base seat about the first axial line, a mount seat that has a second axial line, and a second link that is pivoted to said first link, that is mounted to said mount seat, and that is rotatable relative to said mount seat about the second axial line;
a drive unit including a first drive motor that is connected to said first link and that is operable to drive rotation of said first link relative to said base seat about the first axial line, and a second drive motor that is connected to said second link and that is operable to drive rotation of said second link relative to said mount seat about the second axial line; and
a control unit electrically coupled to said drive unit, and receiving a control signal to control said first drive motor and said second drive motor to move said first and second links from an initial position to an objective position, such that the relative position between said base seat and said mount seat at the time that said first and second links are at the initial position and the relative position between said base seat and said mount seat at the time that said first and second links are at the objective position are substantially identical to each other.

2. The remote center of motion robotic arm as claimed in claim 1, wherein the first axial line and the second axial line belong to an imaginary plane, a joint between said first and second links moving between two opposite sides of the imaginary plane during the movement of said first and second links from the initial position to the objective position.

3. The remote center of motion robotic arm as claimed in claim 2, further comprising an endoscope that is mounted to said mount seat, and that is oriented in an objective direction for taking images.

4. The remote center of motion robotic arm as claimed in claim 2, wherein the initial position of said first and second links and the objective position of said first and second links are symmetric to each other with respect to the imaginary plane.

5. The remote center of motion robotic arm as claimed in claim 4, further comprising a detection unit that is electrically coupled to said control unit, said detection unit including a first position sensor that is connected to said first drive motor for detecting a rotational position of said first drive motor, and a second position sensor that is connected to said second drive motor for detecting a rotational position of said second drive motor, said control unit calculating an initial relative position among said base seat, said first link, said second link and said mount seat according to the information detected by said first and second position sensors after reception of the control signal so as to determine the initial position of said first and second links, and then calculating an objective relative position among said base seat, said first link, said second link and said mount seat where the position of said first and second links is symmetric to the position of said first and second links at the time that said first and second links are at the initial position with respect to the imaginary plane and where the relative position between said base seat and said mount seat is maintained so as to determine the objective position of said first and second links, to therefore control said first and second drive motors to drive movement of said first and second links from the initial position to the objective position.

6. The remote center of motion robotic arm as claimed in claim 3, wherein the initial position of said first and second links and the objective position of said first and second links are symmetric to each other with respect to the imaginary plane.

7. The remote center of motion robotic arm as claimed in claim 6, further comprising a detection unit that is electrically coupled to said control unit, said detection unit including a first position sensor that is connected to said first drive motor for detecting a rotational position of said first drive motor, and a second position sensor that is connected to said second drive motor for detecting a rotational position of said second drive motor, said control unit calculating an initial relative position among said base seat, said first link, said second link and said mount seat according to the information detected by said first and second position sensors after reception of the control signal so as to determine the initial position of said first and second links, and then calculating an objective relative position among said base seat, said first link, said second link and said mount seat where the position of said first and second links is symmetric to the position of said first and second links at the time that said first and second links are at the initial position with respect to the imaginary plane and where the relative position between said base seat and said mount seat is maintained so as to determine the objective position of said first and second links, to therefore control said first and second drive motors to drive movement of said first and second links from the initial position to the objective position.

8. The remote center of motion robotic arm as claimed in claim 7, further comprising a monitor that is electrically coupled to said endoscope for displaying the images taken from said endoscope.

9. The remote center of motion robotic arm as claimed in claim 5, further comprising a human-machine interface that is electrically coupled to said control unit and that is for emitting the control signal.

10. The remote center of motion robotic arm as claimed in claim 7, further comprising a human-machine interface that is electrically coupled to said control unit and that is for emitting the control signal.
